(19) United States
(12) Patent Application Publication
(10) Pub. No.: US 2024/0374497 A1
(43) Pub. Date: Nov. 14, 2024

(54) COMPOSITIONS COMPRISING OR2AT4 AGONISTS

(71) Applicant: Coty Inc., New York, NY (US)

(72) Inventor: David Joyal, Denville, NJ (US)

(21) Appl. No.: 18/696,874

(22) PCT Filed: Sep. 30, 2022
(86) PCT No.: PCT/US2022/045442

§ 371 Date: Mar. 28, 2024

Related U.S. Application Data
(60) Provisional application No. 63/251,309, filed on Oct. 1, 2021.

(51) Int. Cl. A61K 8/64; A61Q 7/00

(52) U.S. Cl. CPC A61K 8/64; A61Q 7/00; A61K 2800/43; A61K 2800/591

(57) ABSTRACT

Provided herein are compositions, such serums and mascaras or eyeliner, for hair growth, in particular non-scalp hair growth, including increasing the number of visible eyelashes, as well as improving lash shine, volume, and length; and thus, is beneficial for enhancing the appearance of eyelashes. as well as eyebrows (and other facial hair, such as mustaches and beards).

### CROSS-REFERENCE TO RELATED APPLICATIONS

This application claims the benefit of U.S. Provisional Appl. Ser. No. 63/251,309, filed Oct. 1, 2021, which is incorporated by reference as if fully set forth herein.

### BACKGROUND

Consumers wish to improve the appearance of their eyelashes and eyebrows. For example, consumers concern included thinning or sparse eyelashes and/or eyebrows, eyelashes that are not as long as desired, or eyebrows and eyelashes that otherwise lack a healthy, full appearance.

### SUMMARY

Provided herein are compositions, such serums, liquids, ointments, mascaras or eyeliner, for hair growth, in particular non-scalp (the skin covering the head, excluding the face) hair growth, including increasing the number of visible eyelashes, as well as improving lash shine, volume, and length; and thus, is beneficial for enhancing the appearance of eyelashes, as well as eyebrows (and other facial hair, such as mustaches and beards).

On embodiment relates to compositions for topical application to the eyelashes or eyebrows comprising an active ingredient, including an active ingredient of at least one OR2AT4 agonist or an analog thereof.

The compositions may be provided in a cosmetically acceptable vehicle in the form of various eye products, such as a serum, a mascara, or an eyeliner. One embodiment provides a mascara composition that includes an active component for hair growth.

Another embodiment relates to cosmetic use of compositions described herein for improving eyelash and/or eyebrow appearance and/or health. Such improvements can comprise: (a) improvement in lash root sheath thickness; (b) improvement in lash anchorage; (c) decrease in lash loss; (d) reduction in lash breakage; (e) increase in lash strength; (f) improvement in lash growth rate; (g) improvement in lash shine; (h) improvement in the number of visible lashes; i) improvement in lash length; and/or (j) improvement in lash volume.

The compositions can be applied to the eyelashes or eyebrows, including along the eyelash fibers and/or at the base of the lashes to contact the eyelash follicles. In some embodiments, effective amounts of of at least one OR2AT4 are provided in a cosmetically acceptable vehicle and topically applied to eyelash follicles for a time sufficient to produce the desired effect. Lash benefits may be realized immediately following application, or after a period of time of such usage. In some embodiments, the inventive composition is applied to eyelashes follicles at least twice daily and/or for a period of at least 8 or at least 12 weeks.

An embodiment provides kits comprising a composition described herein. Such kits will typically include instructions, directing application of the products in a manner to provide doses for achieving significant improvements in eyelash and/or eyebrow health and/or appearance.

One embodiment provides, for example, a mascara composition or other topical hair composition, for strengthening eyelashes and/or promoting eyelash growth or eyebrow growth. The composition contains an active ingredient of at least an OR2AT4 agonist or an analog thereof. The subject matter of the present invention also includes a cosmetic method for promoting eyelash growth, reducing eyelash loss and/or promoting eyebrow growth.

In one embodiment, an OR2AT4 agonist is included in a mascara formulation, or other eyelash formulations and eyebrow formulations.

In another embodiment a material increasing production of the anagen-prolonging growth factor IGF-1 is included in a mascara formulation.

In another embodiment, metabolites of resident hair follicle microbiota that exhibit OR2AT4-mediated upregulation of IGF-1 and/or dermacidin production are included in a mascara, or other eyelash, and eyebrow formula (Lange-Asschenfeldt et al. Skin Pharmacol Physiol 2011; 24:305-311).

In another embodiment a material decreasing production of the catagen-promoting growth factor TGF-β2 is included in a mascara, or other eyelash, and eyebrow formulation.

One embodiment provides a topical composition comprising: a) an active agent; b) a pigment; c) a continuous phase; and d) a discontinuous phase, wherein the active agent is at least one OR2AT4 agonist, IGF-1 or a combination thereof. Another provides a topical composition comprising at least one OR2AT4 agonist, IGF-1 or combination thereof in a cosmetically acceptable vehicle; wherein said tropical composition is in the form of a serum, a mascara, or an eye liner. In one embodiment, the composition is selected from the group consisting of water in oil, oil in water, water in silicone, or silicone in water emulsions. In another embodiment, the OR2AT4 agonist is selected from (+)-alpha-Santalol; (−)-beta-santalol; sandela; sandacore; bacdanol; sandrox; sandex, sandal butenol sandalore; polysantol; ebanol; osyrol; firsantol; javanol; fleursandol; pashminol; sandal pentanol, sandelwood, sandal cyclopropane, sandal cyclopentane, sandalrome and brahamol.

One embodiment provides a method for improving eyelash or eyebrow appearance comprising topically applying to eyelashes, eyelash follicles and/or eyebrows a composition comprising at least one OR2AT4 agonist, IGF-1 or a combination thereof in a cosmetically acceptable vehicle. In embodiment, the composition is applied at least once daily. In one embodiment, said improvement comprises at least one selected from the group consisting of: (a) improvement in lash roof sheath thickness; (b) improvement in lash anchorage; (c) decrease in lash loss; (d) reduction in lash breakage; (e) increase in lash strength; (f) improvement in lash growth rate; (g) improvement in lash shine; (h) improvement in the number of visible lashes; (i) improvement in lash length; and (j) improvement in lash volume. One embodiment provides a method for improving the visual length, thickness, number, and density of eyelash hair and eyebrow hair comprising applying an effective amount of a topical composition comprising at least one OR2AT4 agonist, IGF-1 or a combination thereof to subject on an area of where hair growth is desired.

One embodiment provides a kit comprising a serum and a mascara, in separate containers, for topical application to eyelashes and/or eyelash follicles, wherein the serum and/or the mascara comprise at least one OR2AT4 agonist in a cosmetically acceptable vehicle. In one embodiment, the OR2AT4 agonist is selected from (+)-alpha-Santalol; (−)-beta-santalol; sandela; sandacore; bacdanol; sandrox; sandex, sandal butenol sandalore; polysantol; ebanol; osyrol; firsantol; javanol; fleursandol; pashminol; sandal pentanol, sandelwood, sandal cyclopropane, sandal cyclopentane, sandalrome and brahamol.

### BRIEF DESCRIPTION OF THE DRAWINGS

FIG. 1 and FIG. 2 depict several compounds for use in the compositions and methods provided herein.

FIG. 3. Bar graph of Sandalwood, firsantol activity.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments; however, it will be appreciated that the scope of the invention may include embodiments having combinations of all or some of the features described.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

According to various aspects of the present disclosure an eyelash composition, eyebrow composition and/or mascara component composition can include an active component. The active component can include an OR2AT4 agonist or analogs thereof. Olfactory receptors are expressed by different cell types throughout the body and regulate physiological cell functions beyond olfaction. In particular, the olfactory receptor OR2AT4 has been shown to stimulate keratinocyte proliferation in the skin. It is believed that specific stimulation of OR2AT4 can prolong eyelash and/or eyebrow hair growth ex vivo by decreasing apoptosis and increasing production of the anagen-prolonging growth factor IGF-1.

Olfactory receptors (ORs) are part of an evolutionarily ancient chemosensory signaling system that long predates the development of smell sensation (olfaction). OR expression is not restricted to the nasal epithelium, but it is also present in several other human tissues. Non-olfactory roles of ORs have also been described in human cell physiology, such as in spermatozoa and enterochromaffin cells of the gut (Cheret et al. Nature Com. 2018. Vol 9).

Interestingly, several ORs are also expressed in human epidermis, including OR2AT4, whose selective activation by the synthetic sandalwood odorant (Sandalore®) promotes human epidermal keratinocyte migration and proliferation in vitro and wound re-epithelialization ex vivo.

### Compositions/Formulations

One embodiment relates to a composition comprising at least one active that act improves eyelash and/or eyebrow appearance and/or health. The composition comprises at least one OR2AT4 agonist.

Sandal pentanol is an example of a OR2AT4 agonist for use in the present compositions and methods. Sandal pentanol is a synthetic odorant having a fragrance used as an ingredient mimicking the sandalwood scent. Sandal pentanol ia an alcohol having a chemical structure quite distinct from the ingredients of natural sandalwood oil which usually are extracted by steam distillation of wood from matured sandalwood trees. The IUPAC denomination of sandal pentanol is 3-methyl-5-(2,2,3-trimethylcyclopent-3-en-1-yl) pentan-2-ol. Sandal pentanol has been shown to have a stimulatory effect on the olfactory receptor OR2AT4, which is expressed in the epithelium of human hair follicles. Simulation of OR2AT4 by sandal pentanol in turn prolongs hair growth ex vivo by decreasing apoptosis and increasing the production of growth factor IGF-1. Sandal pentanol accordingly in believed to act as an agonist promoting the growth of mammalian hair.

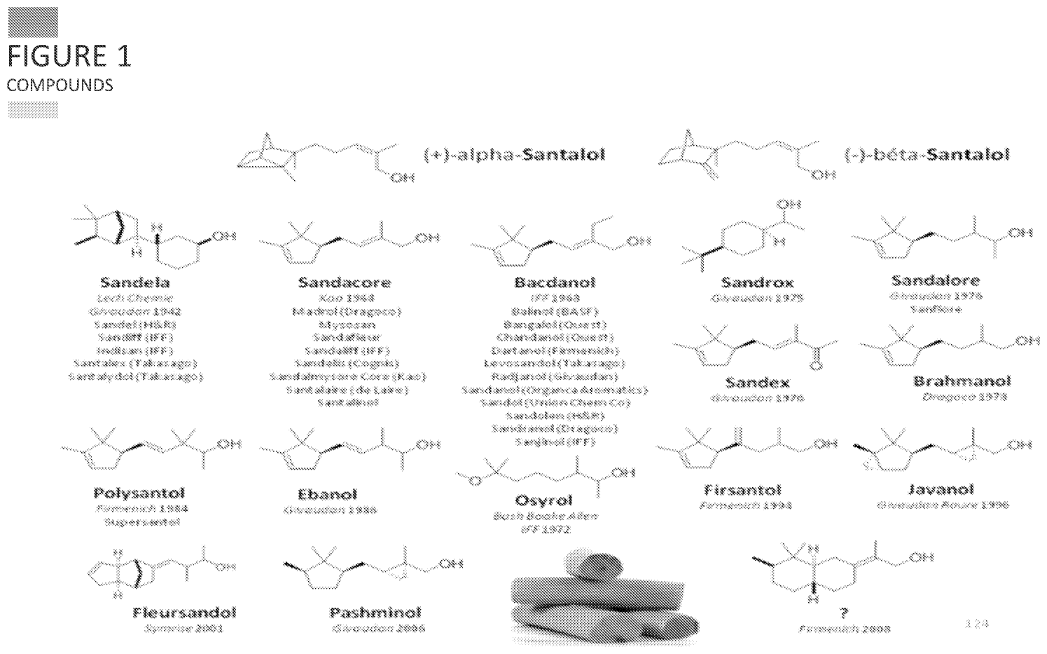

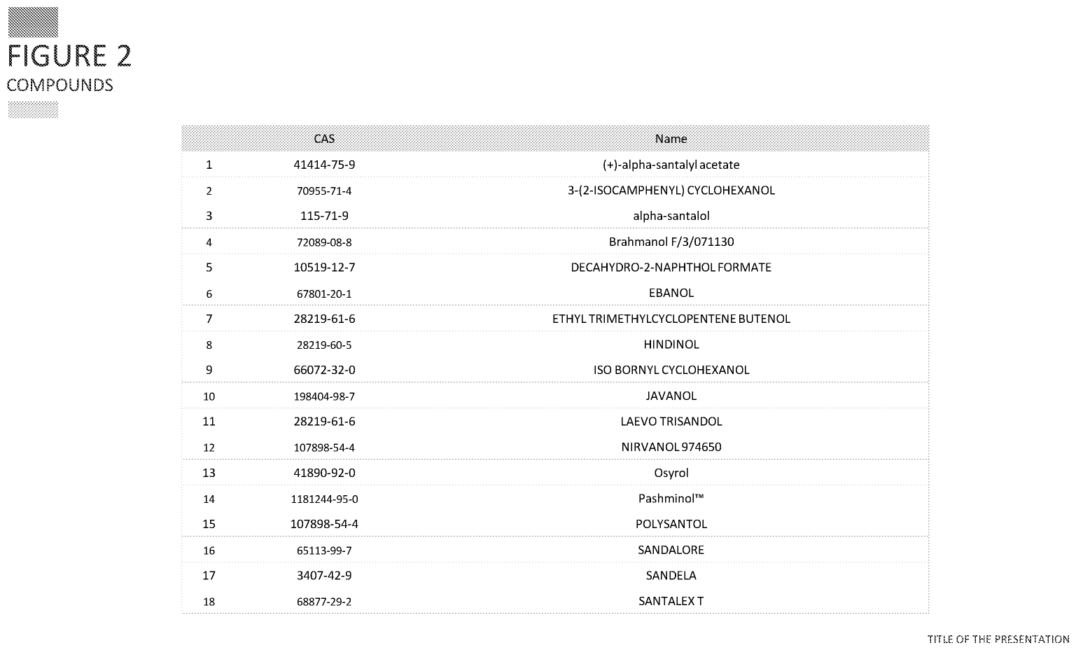

Analogs or derivatives, respectively, of sandal pentanol are believed to have a similar effect on mammalian hair growth, and may be used in the present disclosure alternatively or additionally to sandal pentanol. Sandal pentanol analogs suitable as active ingredients include:
(4Z)-3-methyl-5-(2,2,3-trimethylcyclopent-3-en-1-yl)pent-4-en-2-ol (sandal pentenol)
1-methyl-2-((1,2.2-trimethylbicyclo (3.1.0)hex-3-yl)methyl)-cyclopropane-methanol (sandal cyclopropane)
(E)-3,3-dimethyl-5-(2,2,3-trimethylcyclopent-3-en-1-yl)pent-4-en-2-ol (santol pentenol)
2-methyl-4-(2,2,3-trimethyl-3-cyclopenten-1-yl)butanol (sandal cyclopentane)
(E)-2-ethyl-4-(2,2,3-trimethylcyclopent-3-en-1-yl)but-2-en-1-ol (sandalrome)
(E)-2-methyl-4-(2,2,3-trimethyl-3-cyclopenten-1-yl)-2-buten-1-ol (sandal butenol)
Brahamol
Other hair growth promoting compounds for use in the compositions and claims include: (+)-alpha-Santalol; (−)-beta-santalol; sandela; sandacore; bacdanol; sandrox; sandex, sandalore; polysantol; ebanol; osyrol; firsantol; javanol; fleursandol; pashminol, (R*,R*)-a,4-dimethyl-a-(4-methyl-3-pentenyl) cyclohex-3-ene-1-methanol; 4-penten-1-ol, 2-methyl-4-(2,2,3-trimethyl-3-cyclopenten-1-yl)-(firsantol) (see FIGS. 1, FIG. 2 and Table 1; PCT/EP2017/062110).

TABLE 1
Further compounds for use in the compositions and methods

| | | | | | Formula/ |
|---|---|---|---|---|---|
| | Compound | IUPAC Name | CAS N^{o} | Structural formula | MW |
| 1 | sandal pentanol | 3-methyl-5-(2,2,3- trimethylcyclopent-3-en-1- yl)pentan-2-ol | 65113-99-7 | 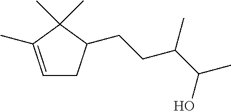 | C₁₄H₂₆O 210.36 |
| 2 | sandal pentenol | (4Z)-3-methyl-5-(2,2,3- trimethylcyclopent-3-en-1- yl)pent-4-en-2-ol | 67801-20-1 | 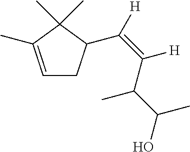 | C₁₄H₂₄O 208.35 |
| 3 | sandal cyclopropane | 1-methyl-2-((1,2,2- trimethylbicyclo(3.1.0)hex-3- yl)methyl)-cyclopropane- methanol | 198404-98-7 | 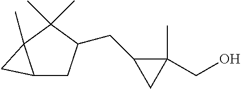 [SMILES: CC1(CO)CC1CC1CC2CC2(C)C1(C)C] | C₁₅H₂₆O 222.37 |
| 4 | Santol pentenol | (E)-3,3-dimethyl-5-(2,2,3- trimethylcyclopent-3-en-1- yl)pent-4-en-2-ol | 107898-54-4 | 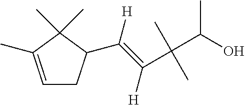 [SMILES: CC1=CCC(/C=C/C(C)(C)C(C)O)C1(C)C] | C₁₅H₂₆O 222.37 |
| 5 | sandal cyclopentane | 2-Methyl-4-(2,2,3-trimethyl-3- cyclopent-1-yl)butanol | 72089-08-8 | 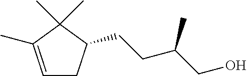 | C₁₃H₂₄O 196.34 |
| 6 | sandalrome | (E)-2-ethyl-4-(2,2,3- trimethylcyclopent-3-en-1-yl)but- 2-en-1-ol | 28219-61-6 | 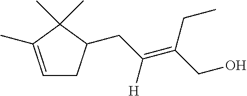 | C₁₄H₂₄O 208.35 |

Cosmetic compositions described herein will comprise an amount of OR2AT4 agonist effective to provide a benefit to eyelashes, such as without limitation improving lash root sheath thickness and/or improving lash anchorage, and/or eyebrows (or other facial hair). For example, the cosmetic composition comprises an amount of an OR2AT4 agonist from 0.4 to about 1.25 weight %, based on the total weight of the composition. Alternatively, the cosmetic composition comprises an amount of OR2AT4 agonist from 1×10⁻⁶ weight % to 3×10⁻⁴ weight %, or about 2×10⁻⁶ weight % to about 2.5×10⁻⁴ weight %, or about 1×10−⁵ weight % to about 2.5×10⁻⁴ weight %, or about 2×10⁻⁵ weight % to about 2×10⁴ weight %, based on the total weight of the composition. In some embodiments, the composition comprises at least about 1×10⁻⁵ weight %, or at least about 2×10⁻⁵, or at least about 1×10⁻⁴ weight % or an OR2AT4 agonist. In some embodiments, the composition comprises at least about 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 μM of an OR2AT4 agonist. In some embodiments, the composition contains no more than 0.03% by weight of the active agent. However treatment according to the present invention can include a very wide effective concentration range of the active agents depending the specific therapeutic goal desired. For example, the active agents (e.g., at least one OR2AT4 agonist) can have concentrations from 0.0075 to 0.03% by weight in liquid mixtures with inert ingredients.

Another embodiment relates to methods for making cosmetic compositions described herein. The OR2AT4 agonist containing compositions can be prepared in any conventional manner, such as by mixing and blending the components in any order. In some embodiments, the components are directly mixed into other cosmetic formulations for topical application to the eyes, eyebrows or eyelashes.

Embodiments provide methods for improving eyebrow and/or eyelash appearance and/or health by topically applying a composition comprising creatine and an OR2AT4 agonist to eyebrow or eyelash fibers and/or follicles for a period of time sufficient to produce one or more of the benefits described herein. Benefits may occur instantly or over the long-term. In some embodiments, a mascara comprising a composition described herein can be applied to the eyelashes. For example, the mascara is applied along the length of the eyelash fiber, optionally including touching the lash base, to contact the lash follicles. Mascara can be applied to top and/or bottom lashes using, e.g., at least about 2, at least about 3, at least about 4, at least about 5, at least about 10, at least about 15, or a least about 20 strokes of a mascara wand, wherein the wand is can be dipped into the mascara product between top and bottom lash applications. The initial coat can be allowed to dry, followed by application of a second coat or additional coats, e.g., in the same or similar manner as the first coat was applied. Mascara may also be “touched up” as needed during wear. In some embodiments, mascara application is used alone or in conjunction with application of a serum (which also can be used alone and can contain an OR2AT4 agonist).

A serum product for topical application to the eyebrows or eyelashes can be used. For example, in some embodiments, a serum comprising a composition described herein can be applied to the upper and/or lower lash line and eyebrows and lashes. The serum is typically clear or colorless. When using it in combination with mascara, the serum can be allowed to dry, e.g., before applying mascara. Application can be repeated one, two, three or more times daily, for as long as is necessary to achieve desired results, such as brow or lash nourishing and/or volumizing effects. The serum can applied at least twice daily, e.g., once in the morning, such as before applying mascara; and once in the evening, such as after removing mascara at night.

In some embodiments, the use or application of the compositions provided herein avoids application to the scalp or hair of the scalp.

Based on the instant disclosure, one of skill in the art will recognize other cosmetic and/or personal care applications for the compositions described herein, and such applications are also contemplated as within the scope of the instant invention. For example, compositions described herein may also find use in eye creams or face creams for day or night use that will be topically applied to areas including the eyelids and eyelash follicles, eyebrows, mustache and beard. The cosmetic compositions described herein generally find use in various eye products, such as serum, mascara, or eyeliner, formulated for topical application to the lashes and/or lash follicles, eyebrows and face, e.g., with a cosmetically acceptable vehicle.

The compositions described herein can be formulated as a variety of eye products for topical application to the eyebrows or lashes. The composition may be formulated in a variety of product forms suitable for application to the eyebrows or lashes, such as, for example, a lotion, cream, serum, spray, aerosol, cake, ointment, essence, gel, paste, patch, pad, eyelash strip, pencil, pomade, solution, towelette, mask, stick, foam, elixir, mousse, powder, foaming cleanser, concentrate, or any other liquid, semi-solid, or solid form. In one embodiment, the composition is formulated as a serum, mascara or eye liner, e.g. for topical application to the eyelash including the base of the eyelash.

The compositions can include, a cosmetically acceptable vehicle. A cosmetically acceptable vehicle refers to any vesicle, for a cosmetic, drug or medicament that is suitable for use in direct, safe contact with human tissues and/or human hair, including human eyebrows, eyelids and eyelashes, and may include, e.g., any diluent, solvent, carrier, filler, or the like. Such vehicles may take the form of any known in the art suitable for application to skin and/or hair, and may include, without limitation, hydrocarbons, glycerin, C1-4 alcohols, fatty alcohols, fatty ethers, fatty esters, polyols, glycols, vegetable oils, mineral oils, liposomes, laminar lipid materials, silicone oils, and any combinations thereof.

The vehicle may comprise an aqueous phase, an oil phase, an alcohol phase, a silicone phase, individually or as mixtures thereof. The cosmetically acceptable vehicle may also comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like, for example, having the appearance of a cream, gel or micro-emulsions. The emulsion may include an emulsifier, such as a nonionic, anionic or amphoteric surfactant.

The aqueous phase of the emulsion may include water, as well as one or more additional solvents, including lower alcohols, such as ethanol, isopropanol, and the like. The volatile solvent may also be a cosmetically acceptable ester such as butyl acetate or ethyl acetate; ketones such as acetone or ethyl ethyl ketone; or the like.

The oil phase of the emulsion has one or more organic compounds, including emollients; humectants (such as butylene glycol, propylene glycol, Methyl gluceth-20, and glycerin); other water-dispersible or water-soluble components including thickeners such as veegum or hydroxyalkyl cellulose; gelling agents, such as high MW polyacrylic acid, i.e. CARBOPOL 934; and mixtures thereof. The emulsion may have one or more emulsifiers capable of emulsifying the various components present in the composition.

The oil phase may comprise one or more waxes, including for example, rice bran was, carnauba wax, ouricurry wax, candelilla wax, montan waxes, sugar cane waxes, ozokerite, polyethylene waxes, Fischer-Tropsch waxes, beeswax, botanical waxes, microcrystalline wax, silicone waxes, fluorinated waxes, and any combination thereof. The oil phase may comprise one or more volatile and/or non-volatile silicone oils.

Based on the disclosures herein, a person skilled in the art will be able to select any of these vehicles, or any other materials described herein, and/or an amount thereof, and/or a proportion thereof, such that the eyebrow, lash nourishing and/or lash enhancing properties of the compositions of the instant invention can be conserved.

The composition may comprise one or more colorants. Suitable colorants, including pigments, lakes, and dyes, are well known in the art and are disclosed in the International Cosmetic Ingredient Dictionary and Handbook, 11^{th} Edition, 2006 (“INCI”), the contents of which are hereby incorporated by reference. Organic pigments can include, for example, FD&C dyes, D&C dyes, including D&C Red, Nos. 2, 5, 6, 7, 10, 11, 12, 13, 30 and 34, D&C Yellow No. 5, Blue No. 1, Violet No. 2. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe2O3, β-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Other suitable colorants include ultramarine blue (i.e., sodium aluminum silicate containing sulfur),

Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, and the like. Further, one or more chroma-methicone colorants may be used, e.g., chromalite yellow-methocone, chroma-life red-methicone, and chroma-lite black-methicone. Whereas serum products are typically clear and colorless, mascara products are typically colored, and in particular black, brown, brown/black, and navy shades are contemplated for use with a mascara comprising a composition described herein.

The inventive compositions may optionally include preservatives. When present, the preservatives will include from about 0.01 to about 5% by weight, typically about 0.05 to about 4% by weight, and preferably about 0.1 to about 3% by weight % of the total composition.

In some embodiments, the compositions of the invention comprise viscosity and/or pH modifiers. Those of skill in the art will recognize that the pH and/or viscosity can be controlled/altered by adding selected pH and/or viscosity modifiers.

The compositions of the invention may optionally comprise other active and inactive ingredients typically associated with cosmetic and personal care eye products, including, but not limited to, excipients, fillers, emulsifying agents, antioxidants, surfactants, additional film-formers, chelating agents, gelling agents, thickeners, emollients, humectants, moisturizers, vitamins, sodium ascorbyl/cholesteryl phosphate, minerals, viscosity and/or theology modifiers, sunscreens, keratolyics, retinoids, hormonal compounds, alpha-hydroxy acids, trioxaundecanedioic acid, alpha-keto acids, anti-mycobacterial agents, antifungal agents, antimicrobials, antivirus, analgesics, lipidic compounds, anti-allergenic agents, H1 or H2 antihistamine, anti-inflammatory agents, anti-irritants, antineoplastics, immune system boosting agents, immune system suppressing agents, anesthetics, antiseptics, skin cooling compounds, skin protectants, skin penetration enhancers, exfoliants, lubricants, fragrances, additional colorants, staining agents, preservatives, stabilizers, pharmaceutical agents, photo-stabilizing agents, optical diffusers, UV absorbers, UV blockers, conditioners, and the like, and mixtures thereof. Based on the teachings herein, a person skilled in the art will be able to select any of these active or inactive ingredients, or any other materials described herein, and/or an amount thereof, and/or a proportion thereof, such that the desirable properties of the cosmetic compositions of the instant invention can be conserved.

In some embodiments, two or more different product forms may be provided together, e.g., as a kit. In certain embodiments, a kit is provided comprising a serum for topical application to eyelashes, and a mascara for topical application to eyelashes (e.g., including eyelash fibers and follicles), where the serum and mascara each comprise a composition according to the instant invention, in a cosmetically acceptable vehicle. In some embodiments, the kit further comprises an eyeliner comprising a composition described herein in a cosmetically acceptable vehicle. In some other embodiments, the eye liner may replace the serum or mascara product or the kit. Such kits will typically also include instructions for use.

### Exemplary Mascara Formulation to Which Compounds Described Herein, Such as an OR2AT4 Against, Can be Added

The compounds disclosed herein can be used in mascara formulations, such as an oil-in-water mascara formulation that includes non-emulsifying wax(es) in a concentration of 0.10 to 12.00 percent by weight; at least one viscosity modifier; and at least one non-wax grafted polymer and at least one wax grafted polymer with a combined concentration of 0.10 percent to 50.00 percent. Mascara formulations within which the compounds disclosed herein, including OR2AT4 agonists, may also be incorporated include those generally described below:

TABLE 2
An oil-in-water mascara formulation, comprising:

| | |
|---|---|
| STAGE 1 | |
| CERA CARNAUBA/COPERNICIA CERIFERA | 2.00% |
| (CARNAUBA) WAX/CIRE DE CARNAUBA | |
| BEESWAX | 8.00% |
| STEARIC ACID | 1.00% |
| CETYL ALCOHOL | 2.00% |
| VP/EICOSENE COPOLYMER | 3.00% |
| STAGE 2 | |
| WATER | QS |
| HECTORITE | 0.30% |
| HYDROXYETHYLCELLULOSE | 0.20% |
| PROPYLENE GLYCOL | 3.00% |
| WATER (and) DISODIUM EDTA | 0.10% |
| STAGE 3 | |
| WATER (and) TETRASODIUM EDTA | 0.12% |
| STAGE 4 | |
| IRON OXIDES | 2.00% |
| IRON OXIDES | 5.00% |
| WATER (and) POLYSORBATE 20 | 1.60% |
| STAGE 5 | |
| AMINOMETHYL PROPANEDIOL | 0.30% |
| STAGE 6 | |
| HYDROGENATED PALM GLYCERIDES (and) POTASSIUM | 2.60% |
| CETYL PHOSPHATE | |
| STAGE 7 | |
| PHENOXYETHANOL | 0.80% |
| WATER (and) PANTHENOL (and) PANTOLACTONE | 0.80% |
| TOCOPHERYL ACETATE | 0.20% |
| ARGANIA SPINOSA KERNEL OIL | 0.10% |
| HYDROLYZED KERATIN (and) WATER (and) POTASSIUM | 0.03% |
| SORBATE (and) SODIUM BENZOATE (and) | |
| PHENOXYETHANOL (and) SODIUM SULFATE | |
| COLLAGEN (and) ACRYLATES/CARBAMATE | 0.03% |
| COPOLYMER | |
| STAGE 8 | |
| AMMONIUM ACRYLATES COPOLYMER (AND) WATER | |
| (AND) ALCOHOL DENAT. (AND) DISODIUM DECETH-6 | |
| SULFOSUCCINATE (AND) LAURETH-30 (AND) SODIUM | 30.00% |
| DEHYDROACETATE | |
| ETHYLENE/ACRYLIC ACID COPOLYMER and | 20.00% |
| STYRENE/ACRYLATES COPOLYMER and WATER and | |
| PHENOXYETHANOL and CAPRYLYL GLYCOL | |
| and SODIUM LAURETH-12 SULFATE and | |
| C11-15 PARETH-7 and C11-5 PARETH-40 | |

The particular mascara formulation and other inventive embodiments disclosed herein include an oil-in-water mascara formulation that includes a non-emulsifying wax in a concentration of 0.10 to 12.00 percent by weight; at least one viscosity modifier; and at least one non-wax grafted polymer and at least one wax grafted polymer with a combined concentration of 0.1 percent to 50 percent. While the ammonium acrylates copolymer formulation concentration is listed as 30.00% by weight and the styrene/acrylates copolymer formulation is listed as 20.00% by weight above, it is understood that other concentration ranges are suitable so long as the total concentration of these two formulations is 50% of the mascara formulation.

These ingredients form a strong, yet volumizing film, which eliminates the need for very high concentrations of waxes in the formula. Mascara formulation embodiments also include 12% or less of non-emulsifying wax in an oil phase, about 50% less than what is used conventionally in high volume washable mascara formulations. This combination of ingredients work in synergy, to form a durable and long lasting, yet high volumizing film, which is easily removed with soap and water or warm water alone. Some embodiments are easily removed with soap and water. Other embodiments are easily removed with warm water alone.

Inventive mascara embodiments directly solve a problem of making a long-lasting mascara that is also easily removable with soap and water or with warm water alone, without soap.

TABLE 3
An oil-in-water mascara, comprising:

| | Concentration |
|---|---|
| INCI Nam | Range (wt. %) |
| Phase A | |
| Copernicia Cerifera (Carnauba) Wax/CIRE DE | 0.1-12 |
| Carnauba | |
| Beeswax | 6.4-9.6 |
| Stearic Acid | 0.1-20 |
| Cetyl Alcohol | 0.1-20 |
| VP/Eicosene Copolymer | 2.4-3.6 |
| Phase B | |
| Water | QS |
| Hectorite | 0.1-5 |
| Hydroxyethylcellulose | 0.1-5 |
| Propylene Glycol | 2.4-3.6 |
| Water and Disodium EDTA | 0.08-0.12 |
| Phase C | |
| Water and Tetrasodium EDTA | 0.096-0.144 |
| Phase D | |
| Iron Oxides | 0.1-15 |
| Water and Polysorbate 20 | 1.28-1.92 |
| Phase E | |
| Aminomethyl Propanediol | 0.24-0.36 |
| Phase F | |
| Hydrogenated Palm Glycerides (and) Potassium Cetyl | 0-20 |
| Phosphate | |
| Phase G | |
| Phenoxyethanol | 0.64-0.96 |
| Water (and) Panthenol (and) Pantolactone | 0.64-0.96 |
| Phase H | |
| Ammonium Acrylates Copolymer (and) Water (and) | 15-30 |
| Alcohol Denat. (and) Disodium Deceth-6 | |
| Sulfosuccinate (and) Laureth-30 (and) Sodium | |
| Dehydroacetate | |
| Ethlene/Acrylate Acid Copolymer and | 5-20 |
| Styrene/Acrylates Copolymer and Water and | |
| Phenoxyethanol And Caprylyl Glycol and Sodium | |
| Laureth-12 Sulfate and C(11-15) Pareth-7 and C(11-5) | |
| Pareth040 | |

While hectorite and hydroxymethylcellulose are disclosed as aqueous viscosity modifiers, it is believed that other aqueous viscosity modifiers may be usable in formulations disclosed herein, either singly or in combination.

While stearic acid and cetyl alcohol are disclosed herein as emulsifiers, it is believed that other emulsifiers including hydrogenated palm glycerides and potassium cetyl phosphate, and polysorbate 20, and other conventional emulsifiers are also suitable emulsifiers used singly or in combination.

While black iron oxide is disclosed herein, it is believed that other pigments are usable in formulation embodiments disclosed herein. Specific black iron oxide formulations employed include Black Iron Oxide C33-5000 and black iron oxide C33-6000, both manufactured by Sun Chemical.

While aminomethyl propandiol is disclosed herein, it is believed that other conventional pH adjusters and buffers are usable for use herein.

For some embodiments, the concentration of ingredients in Phase H exceeds 50% by weight.

The term, “QS” used to describe the concentration of water refers to the amount of water added to take the formulation to 100% by weight.

Embodiments disclosed herein additionally include film formers. In the formulation embodiment disclosed herein, the polymer combination includes at least one polyolefin grafted film former and one or more acrylates type film former, in a combined concentration of 0.1-50%.

In the exemplary formulation embodiment disclosed herein, a non-emulsifying wax is Carnauba wax, listed in Stage 1. Carnauba wax is also known as Carnauba Wax Prime, Yellow 2442, manufactured by Kahl GmbH &Co. Another wax is the 8104 Kahl White Beeswax, manufactured by Kahl GmbH & Co. The stearic acid and cetyl alcohol (Lanette 16) are emulsifiers that enable stage 1 ingredients to blend with aqueous based ingredients in Stage 2. VP/Eicosene copolymer, sold as Unimer U-15, manufactured by Induchem USA, is alkylated vinyl pyrolidone copolymer. In the formulation embodiments disclosed herein, the alkylated vinyl pyrolidone acts as a film former and pigment dispersing agent.

Stage 2 is an aqueous stage with water, a powdered smectite clay, hectorite, sold as Bentone EW, manufactured by Element is Specialties; hydroxyethyl cellulose, sold as NATROSOL 250 HR PC, manufactured by Ashland Inc. Stage 2 also includes propylene glycol and water.

The mascara pigments, black iron oxide, are ingredients in Stage 4 of the formulation.

Stage 5 includes the aminomethyl propanediol, sold as AMPD Ultra-PC, which acts to neutralize acids in the formulation to form salts and water.

The phenoxyethanol of Stage 7 is a germicide. D-Panthenol 75L, manufactured by Coptis, is a moisturizer. Vitamin E is an antioxidant.

For some embodiments, other waxes are used in conjunction with non-emulsifying waxes. The low concentrations of non-emulsifying waxes provide a flexible formulation, while also adding to the building characteristics of the formula by providing structure and slip necessary to aid in volumizing benefit of mascara.

Volumizing mascara embodiments disclosed herein provide pristine wear and easy removal from eyelashes with soap and water for some embodiments or warm water alone as described herein. Formulation embodiments disclosed herein provide an optimized combination of film formers and waxes producing a flexible film that instantly build volume and length and stays put through humid conditions while being easy to remove.

The combination of hectorite, the Bentone smectite clay, and hydroxyethylcellulose act as an aqueous viscosity modifying agent, which in turn, facilitates deposition of the formulation embodiments onto lashes.

Emulsifiers are used in both the oil and water phases to keep the two phases together and to prevent phase separation by forming a tight, stable oil-in-water emulsion.

The combination of two non-wax grafted and wax grafted polymers at a combined concentration of 0.1%-50% aid to form a durable, flexible, and volumizing film that provides additional structure to the formulation as well. Examples of such polymers are formulations that include ethylene/acrylate acid copolymer and styrene/acrylates copolymer and water and phenoxyethanol and caprylyl glycol and sodium laureth-12 sulfate and C(11-15) pareth-7 and C(11-5) pareth-40, with a formulation of ammonium acrylates copolymer, water, alcohol, denatured, disodium deceth-6 sulfosuccinate, laureth-30 and sodium.

By using varying concentration ratios of both a formulation that includes ethylene/acrylate acid copolymer and styrene/acrylates copolymer and water and phenoxyethanol and caprylyl glycol and sodium laureth-12 sulfate and C(11-15) pareth-7 and C(11-5) pareth-40, and a formulation of ammonium acrylates copolymer, water, alcohol, denatured, disodium deceth-6 sulfosuccinate, laureth-30 and sodium of Stage 8, in combination with 0.10-12.00% wax(es), and aqueous viscosity modifying agents, the mascara features of longevity on a lash and volumizing on the lash are achieved. The volumizing mascara formula employs a high concentration of polymer and low concentration of waxes, contrary to known oil in water mascara formulations.

In the embodiments presented herein, the formulation of ammonium acrylates copolymer, water, alcohol, denatured, disodium deceth-6 sulfosuccinate, laureth-30 and sodium has a concentration of 25.00% of the mascara formulation. The formulation that includes ethylene/acrylate acid copolymer and styrene/acrylates copolymer and water and phenoxyethanol and caprylyl glycol and sodium laureth-12 sulfate and C(11-15) pareth-7 and C(11-5) pareth-40, has a concentration of 15.00% of the mascara formulation. The formulation of ammonium acrylates copolymer, water, alcohol, denatured, disodium deceth-6 sulfosuccinate, laureth-30 and sodium is an ammonium acrylates copolymer manufactured by Worlee of Hamburg Germany under the name, Micromer C60/42. The formulation that includes ethylene/acrylate acid copolymer and styrene/acrylates copolymer and water and phenoxyethanol and caprylyl glycol and sodium laureth-12 sulfate and C(11-15) pareth-7 and C(11-5) pareth-40, is manufactured by Interpolymer under the name, Syntran PC 5288, and is formed by grafting an amorphous functional acrylic polymer section onto a crystalline polyethylene backbone.

### EXAMPLE

The following example is provided in order to demonstrate and further illustrate certain embodiments and aspects of the present invention and are not to be construed as limiting the scope thereof.

### Example I

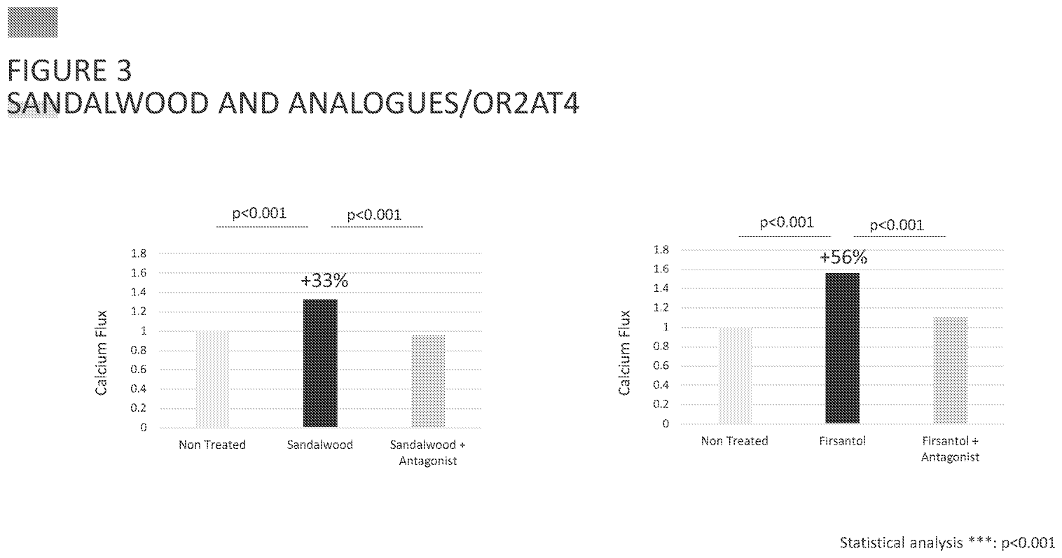

OR2AT4 can be activated, and thereby confer beneficial eyebrow and eyelash properties when applied to eyebrows and eyelashes (or other facial hair), by OR2AT4 agonists as disclosed herein. Normal Human Keratinocyte (NHK) cells were incubated with sandalwood (500 μM) or firsantol (500 μM; Fura-2 AM (Fura-2-acetoxymethyl ester) for 30 minutes (min) at 37° C. After which, the cells were rinsed with Krebs-Ringer solution and incubated for 30 min at 37° C. Assays of the OR2AT4 agonists, with and without antagonist (500 μoxyphenylon), were performed and fluorescence was measured (excitation at 340 nm and 380 nm, emission 510 nm). The results are depicted in FIG. 3.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. A topical composition comprising:
a) an active agent;
b) a pigment;
c) a continuous phase; and
d) a discontinuous phase,
wherein the active agent is at least one OR2AT4 agonist, IGF-1 or a combination thereof.

2. A topical composition comprising at least one OR2AT4 agonist, IGF-1 or combination thereof in a cosmetically acceptable vehicle; wherein said tropical composition is in the form of a serum, a mascara, or an eye liner.

3. The composition of claim 1, wherein the composition is selected from the group consisting of water in oil, oil in water, water in silicone, or silicone in water emulsions.

4. The composition of claim 1, wherein the OR2AT4 agonist is selected from (+)-alpha-Santalol; (−)-beta-santalol; sandela; sandacore; bacdanol; sandrox; sandex, sandal butenol sandalore; polysantol; ebanol; osyrol; firsantol; javanol; fleursandol; pashminol; sandal pentanol, sandelwood, sandal cyclopropane, sandal cyclopentane, sandalrome and brahamol.

5. A method for improving eyelash or eyebrow appearance comprising topically applying to eyelashes, eyelash follicles and/or eyebrows a composition comprising at least one OR2AT4 agonist, IGF-1 or a combination thereof in a cosmetically acceptable vehicle.

6. The method according to claim 5, wherein said composition is applied at least once daily.

7. The method according to claim 5, wherein said improvement comprises at least one selected from the group consisting of: (a) improvement in lash roof sheath thickness; (b) improvement in lash anchorage; (c) decrease in lash loss; (d) reduction in lash breakage; (e) increase in lash strength; (f) improvement in lash growth rate; (g) improvement in lash shine; (h) improvement in the number of visible lashes; (i) improvement in lash length; and (j) improvement in lash volume.

8. A method for improving the length, thickness, number, and density of eyelash hair and eyebrow hair comprising applying an effective amount of the topical composition of claim 1, comprising at least one OR2AT4 agonist, IGF-1 or a combination thereof to subject on an area of where hair growth is desired.

9. A kit comprising the topical composition of claim 1, for topical application to eyelashes and/or eyelash follicles, wherein the serum and/or the mascara comprise at least one OR2AT4 agonist in a cosmetically acceptable vehicle.

10. The method of claim 1, wherein the OR2AT4 agonist is selected from (+)-alpha-Santalol; (−)-beta-santalol; sandela; sandacore; bacdanol; sandrox; sandex, sandal butenol sandalore; polysantol; ebanol; osyrol; firsantol; javanol; fleursandol; pashminol; sandal pentanol, sandelwood, sandal cyclopropane, sandal cyclopentane, sandalrome and brahamol.
